(19) United States
(12) Patent Application Publication
(10) Pub. No.: US 2008/0188914 A1
(43) Pub. Date: Aug. 7, 2008

(54) Detachable handpiece

(75) Inventor: Morgan Lars Ake Gustavsson, Newport Beach, CA (US)

Correspondence Address: PROSKAUER ROSE LLP, ONE INTERNATIONAL PLACE, BOSTON, MA, 02110, US

(73) Assignee: Candela Corporation, Wayland, MA (US)

(21) Appl. No.: 11/800,663

(22) Filed: May 7, 2007

Related U.S. Application Data
(60) Provisional application No. 60/898,927, filed on Feb. 1, 2007.

(51) Int. Cl. A61N 5/00

(52) U.S. Cl. 607/101

(57) ABSTRACT

The invention features a detachable handpiece for treating biological tissue using electromagnetic radiation. The handpiece includes a connector adjacent a first portion of the handpiece, for detachably connecting the handpiece to an umbilicus and for receiving energy from a power conduit in the umbilicus. The handpiece also includes a source of electromagnetic radiation adjacent a second portion of the handpiece and in communication with the connector, for receiving energy from the connector to drive the source of electromagnetic radiation.

### CROSS-REFERENCE TO RELATED APPLICATION

This application claims the benefit of and priority to U.S. Provisional Application Ser. No. 60/898,927 filed Feb. 1, 2007, which is owned by the assignee of the instant application and the disclosure of which is incorporated herein by reference in its entirety.

### FIELD OF THE INVENTION

The invention relates generally to apparatuses including detachable and interchangeable handpieces for treating biological tissue using electromagnetic radiation. The invention relates more particularly to a handpiece that detaches at the handpiece end of an umbilicus and related connectors, which can be included in an apparatus for treating biological tissue using electromagnetic radiation.

### BACKGROUND OF THE INVENTION

Electromagnetic radiation has a wide range of applications in treating biological tissue. For example, electromagnetic radiation has been employed in treatments as broad and varied as treatments for acne, fat, cellulite, oily skin, pigmented lesions, pores, scarring, vascular lesions, and wrinkles, as well as for skin rejuvenation, hair removal, and tattoo removal. However, different treatments can necessitate a wide range of parameters, which can in turn necessitate a wide range of equipment. Obtaining, storing, and maintaining a wide range of equipment can be expensive and impractical.

Different approaches to reducing the expense and increasing the practicality of obtaining, storing, and maintaining the wide range of equipment necessary for implementing broad and varied treatments exists. For example, some apparatuses can have a base unit and handpiece connected by an umbilicus. Multiple handpieces can be provided that are suitable for various treatments. Each handpiece is attached to an umbilicus, and each umbilicus can be attached and reattached to the base unit to substitute a different handpiece.

Some prior art apparatuses include a handpiece connected by an umbilicus to a connector interface. The umbilicus includes conduits for power, signal, and fluid/gas to travel between the handpiece and base unit. The connector interface attaches to a base unit. Thus, changing the handpiece requires removing the handpiece and umbilicus at the base unit. The connector interface includes a power plug, a signal plug, and four fluid/gas plugs. The power plug and signal plug each include multiple pins. In various embodiments, the connector interface can also include other plugs. The connector requires separate conduits or plugs for each function (e.g., power, signal, and fluid transport), which results in a relatively large connector interface.

A handpiece that only disconnects from the base unit by disconnecting the umbilicus from the base unit can be expensive and impracticable. For example, multiple large, unwieldy umbilicus/handpiece combinations must be stored in a practitioner's office, which generally has limited storage space. The point of connection between the base unit and umbilicus is also large because it must accommodate multiple conduits for providing power, signal, and coolant to the handpiece. Separate conduits are used to transmit power and signal.

### SUMMARY OF THE INVENTION

In various embodiments, the invention features a detachable handpiece in an apparatus for treating biological tissue using electromagnetic radiation. The invention also features handpieces that detach at the handpiece end of an umbilicus and the related connectors. The invention can eliminate the need for a signal connector in addition to a power connector. Thus, the connector can be more compact than connectors that require both power and signal connectors. There is no longer the need to detach the umbilicus from the base unit each time a different handpiece is needed.

In some embodiments, the invention features a radio connector for a detachable handpiece, which can communicate a radio signal through two power cords. However, the invention is not limited to communicating a radio signal through two power cords and can include other means to eliminate the need for a signal connector in addition to a power connector. One advantage of the invention is a connector for a detachable handpiece that is smaller than conventional connectors. Another advantage of the invention is a connector that can be used at the handpiece end of the umbilicus rather than at the base unit end, and thus can eliminate the need to purchase, maintain, and store multiple umbilici with multiple handpieces. Other advantages of the invention include cheaper, smaller, and more practical apparatuses for treating biological tissue using electromagnetic radiation.

In one aspect, the invention features a detachable handpiece for treating biological tissue using electromagnetic radiation. The handpiece includes a connector and a source of electromagnetic radiation. The connector is adjacent a first portion of the handpiece, detachably connects the handpiece to an umbilicus, and receives energy from a power conduit in the umbilicus. The source of electromagnetic radiation is adjacent a second portion of the handpiece and is in communication with the connector to receive energy from the connector to drive the source of electromagnetic radiation.

In another aspect, the invention features a connector for a detachable handpiece for treating biological tissue using electromagnetic radiation. The connector includes a first end and a second end. The first end is adapted to detachably connect to an umbilicus having an energy conduit and can receive energy from the energy conduit. The second end is adjacent the handpiece and can communicate energy to the handpiece to drive a source of electromagnetic radiation.

In yet another aspect, the invention features an apparatus for treating biological tissue using electromagnetic radiation. The apparatus includes an energy source, and umbilicus, a first connector, a second connector, and a handpiece. The umbilicus connects to the base unit. The umbilicus has a conduit for transmitting energy from the energy source from a first end to a second end of the umbilicus. The first connector is associated with the second end of the umbilicus. The second connector can detachably connect to the first connector and for receiving the energy. The handpiece is associated with the second connector. The handpiece has a source of electromagnetic radiation in communication with the second connector, for receiving the energy to drive the source of electromagnetic radiation.

In other examples, any of the aspects above, or any apparatus or method described herein, can include one or more of the following features.

In various embodiments, the detachable handpiece can include a controller in communication with the source of electromagnetic radiation, for at least one of sending and receiving a signal and for regulating the source of electromagnetic radiation to deliver electromagnetic radiation to the biological tissue. The controller can be in communication with the connector, for at least one of sending and receiving a signal transmitted through the connector. The signal transmitted through the connector can be an RF signal transmitted through the power conduit. The controller can be adapted for at least one of sending and receiving a wireless signal. The controller can be adapted for receiving an electronic signal.

In some embodiments, the first end of the connector is adapted for at least one of sending and receiving a signal from the umbilicus and the second end is adapted for communicating the signal at least one of to and from the handpiece, to regulate the source of electromagnetic radiation. The signal can be an RF signal transmitted through the power conduit and/or an electronic signal transmitted through a second conduit.

In certain embodiments, the apparatus includes a signal source for providing a signal for regulating the source of electromagnetic radiation. The apparatus can include a controller associated with the handpiece and in communication with the signal source and the source of electromagnetic radiation, for regulating the source of electromagnetic radiation. The umbilicus can be adapted to facilitate communication between the signal source and the source of electromagnetic radiation. The signal source can be adapted for transmitting and/or receiving a wireless signal and the controller can be adapted for transmitting and/or receiving the wireless signal for regulating the source of electromagnetic radiation. The apparatus can include a controller adapted for regulating at least one property of the energy received by the source of electromagnetic radiation, to regulate the source of electromagnetic radiation.

Other aspects and advantages of the invention can become apparent from the following drawings and description, all of which illustrate the principles of the invention, by way of example only.

### BRIEF DESCRIPTION OF THE DRAWINGS

FIG. 1 shows an apparatus for treating biological tissue using electromagnetic radiation.

FIG. 2 shows an embodiment of the apparatus in which the base unit includes a power source and a control source.

FIG. 3 shows another embodiment of the apparatus in which the base unit includes a power source and a control source.

FIGS. 4A and 4B show a connector for attaching a handpiece and an umbilicus.

FIGS. 5A and 5B show another connector for attaching a handpiece and an umbilicus.

FIGS. 6A and 6B show yet another connector for attaching a handpiece and an umbilicus.

FIG. 7 shows a handpiece disconnected from an umbilicus.

### DESCRIPTION OF THE INVENTION

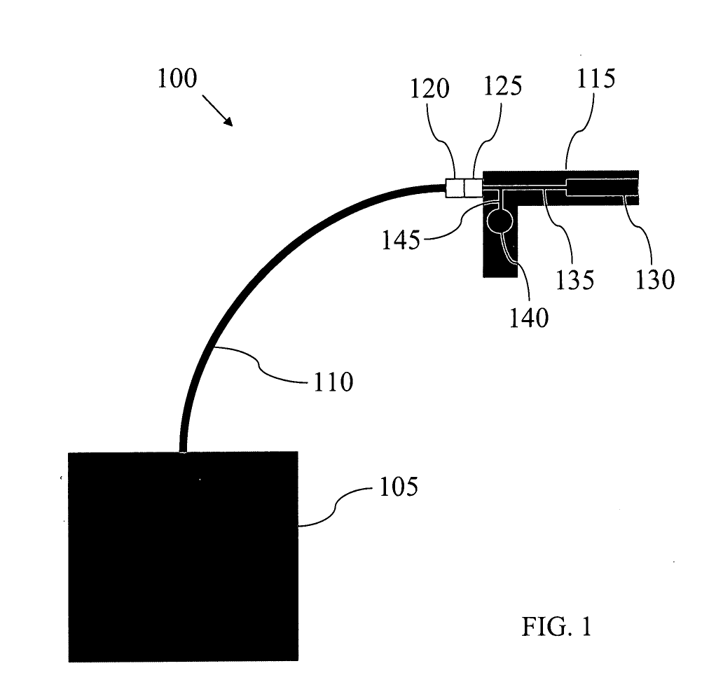

FIG. 1 shows an apparatus 100 for treating biological tissue using electromagnetic radiation. The apparatus 100 includes a base unit 105, an umbilicus 110, and a handpiece 115. The base unit 105 can include a power source, computer, electronics, cooling elements, and/or other components. The umbilicus 110 connects the base unit 105 to the handpiece 115. The umbilicus 110 can include one or more conduits for communicating power, signal, fluid, and/or gas from a first end to a second end, e.g. between the base unit 105 and the handpiece 115. The second end of the umbilicus 110 is associated with a first 120 connector. The handpiece 115 is associated with a second 125 connector that is detachably connectable to the first 120 connector. The handpiece 115 can be in at least one of efferent and afferent signal communication with the base unit 105.

The handpiece 115 also includes a source of electromagnetic radiation 130 adjacent a second portion of the handpiece and in communication with the second 125 connector through a first 135 conduit, for receiving energy from the second 125 connector to drive the source of electromagnetic radiation 130. The handpiece 115 can also include a controller 140 for controlling the source of electromagnetic radiation. The controller 140 is in communication with the source of electromagnetic radiation 130 through a second 135 conduit. In some embodiments, the controller 140 is in communication with the umbilicus 110 through a second 135 conduit. The handpiece 115 can include filters and optics for delivering the electromagnetic radiation to biological tissue. Power can also be used to drive and/or control the source of electromagnetic radiation. A signal can be used to control the output of the source of electromagnetic radiation (e.g., set, maintain, or control parameters of emitted radiation). Cooling elements can include a water pump, a heat exchanger, a thermoelectric cooler, and/or a fan. The controller 140 can send and/or receive the signal. In one embodiment, the output of the source of electromagnetic radiation is controlled by controlling the power delivered to the source of electromagnetic radiation. The fluid and/or gas can be used to cool the source of electromagnetic radiation and/or a transparent or translucent member contacting the skin during treatment.

In certain embodiments, the apparatus includes a signal source for providing a signal for regulating the source of electromagnetic radiation. The apparatus can include a controller associated with the handpiece and in communication with the signal source and the source of electromagnetic radiation, for regulating the source of electromagnetic radiation. The umbilicus can be adapted to facilitate communication between the signal source and the source of electromagnetic radiation. The signal source can be adapted for transmitting and/or receiving a wireless signal and the controller can be adapted for transmitting and/or receiving the wireless signal for regulating the source of electromagnetic radiation. The apparatus can include a controller adapted for regulating at least one property of the energy received by the source of electromagnetic radiation, for regulating the source of electromagnetic radiation.

In various embodiments, the source of electromagnetic radiation can include a fluorescent pulsed light (FPL) or an intense pulsed light (IPL) system. For example, the system can be a LIGHTSTATION™ (by Candela Corporation of Wayland, Mass.), or an OMNILIGHT™, NOVALIGHT™, or PLASMALITE™ system (by American Medical Bio Care of Newport Beach, Calif.). However, the source of electromagnetic radiation can also include a laser, a diode, a coherent light source, an incoherent light source, or any other source of electromagnetic radiation. FPL technologies can utilize laser-dye impregnated polymer filters to convert unwanted energy from a xenon flashlamp into wavelengths that enhance the effectiveness of the intended applications. FPL technologies can be more energy efficient and can generate significantly less heat than comparative IPL systems. A FPL system can be adapted to operate as a multi-purpose treatment system by changing filters or handpieces to perform different procedures. For example, separate handpieces allow a practioner to perform tattoo removal and other vascular treatments. An exemplary FPL system is described in U.S. Pat. No. 5,320,618, the disclosure of which is herein incorporated by reference in its entirety.

In various embodiments, the apparatus for treating biological tissue using electromagnetic radiation can include a cooling system to modulate the temperature in a region of biological tissue and/or minimize unwanted thermal injury to untargeted biological tissue. For example, the cooling system can cool the biological tissue before, during, or after delivery of radiation, or a combination of the aforementioned. Cooling can include contact conduction cooling, evaporative spray cooling, convective air flow cooling, or a combination of the aforementioned.

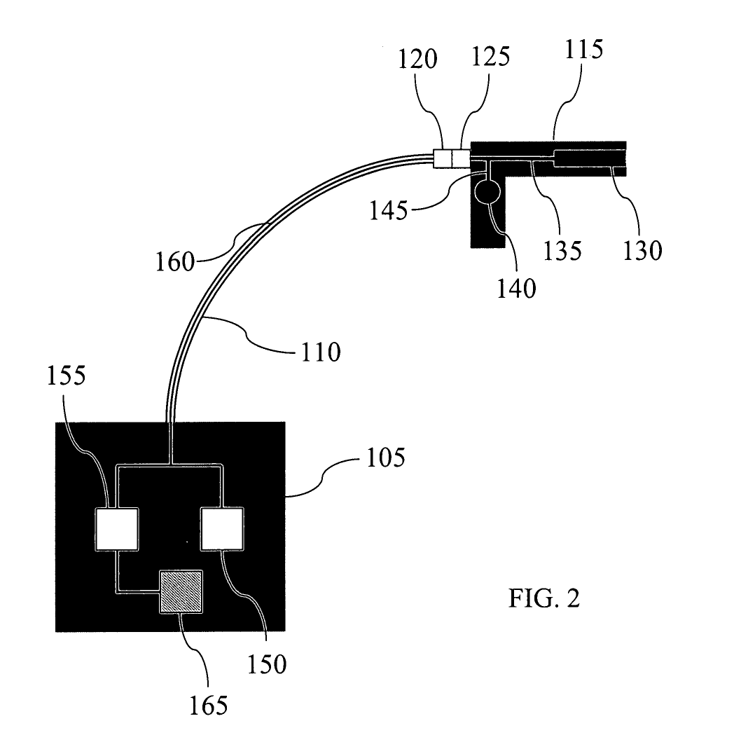

FIG. 2 shows an embodiment of the apparatus 100 in which the base unit 105 includes a power 150 source and a control 155 source. The power 150 source provides power to the handpiece 115 through a conduit 160 in the umbilicus 110. The control 155 source provides a signal to the handpiece 115 through a conduit 160 in the umbilicus 110. The control 155 source can be associated with converter 165, which can convert, transform or otherwise modify the signal. The converter 165 is optional, and can convert a control signal into a signal that can be transmitted through the conduit 160. For example, the converter 165 can convert a digital signal to and/or from an analog signal. In one embodiment, the control signal provided by the control 155 source is transmitted through a first portion of the conduit 160 and the power provided through the power 150 source is transmitted through a second portion of the conduit 160. In another embodiment, the control signal is transmitted through the same portion of the conduit 160 as the power. For example, the control signal can be a radio wave signal transmitted through the same portion of the conduit 160 as the power. The converter 165 can function to convert a signal into a radio wave signal. In various embodiments, the control 155 source can have a user, computer, or network interface.

Both the controller 140 and the control 155 source can send and/or receive the signal. For example, the handpiece 115 can include a trigger, which can be associated with the controller 140, which can initiate the transmission of a signal to the control 155 source (e.g., a signal to start, stop, or modulate the electromagnetic radiation). In various embodiments a signal can be transmitted from the handpiece 115 to the base unit 105 to communicate information regarding a filter for the electromagnetic radiation, the remaining lifetime of the filter, the type of handpiece 115 attached, the band or bands of electromagnetic radiation employed by the handpiece 115, the type of sapphire in the handpiece 115, the spot size of the electromagnetic radiation, and any information useful or necessary for the treatment.

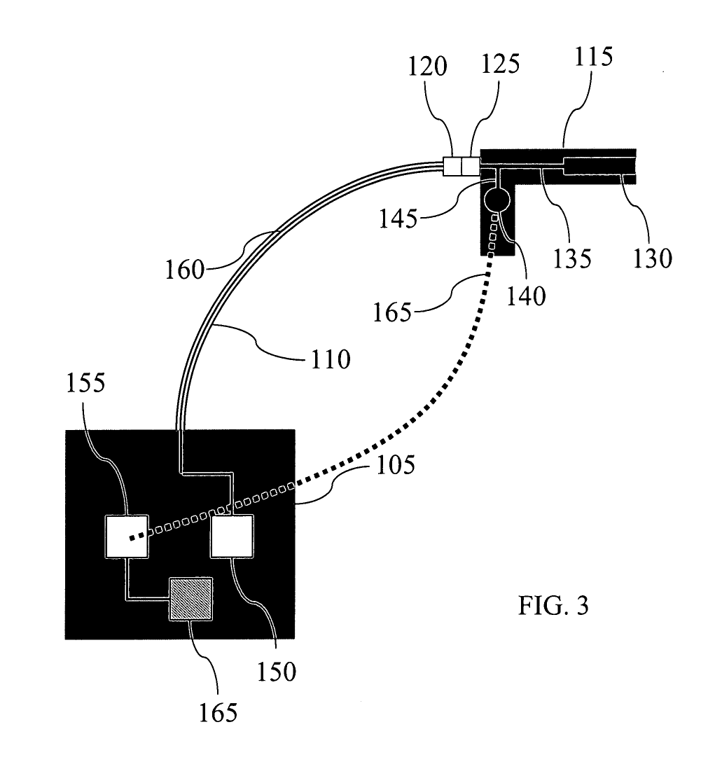

FIG. 3 shows another embodiment of the apparatus 100 in which the base unit 105 includes a power 150 source and a control 155 source. The power 150 source provides power to the handpiece 115 through a conduit 160 in the umbilicus 110. The control 155 source provides a signal to the handpiece 115 through a wireless signal 165. The control 155 source can be associated with converter 165, which can convert, transform or otherwise modify the signal. The converter 165 is optional, and can convert a control signal into a signal that can be transmitted wirelessly.

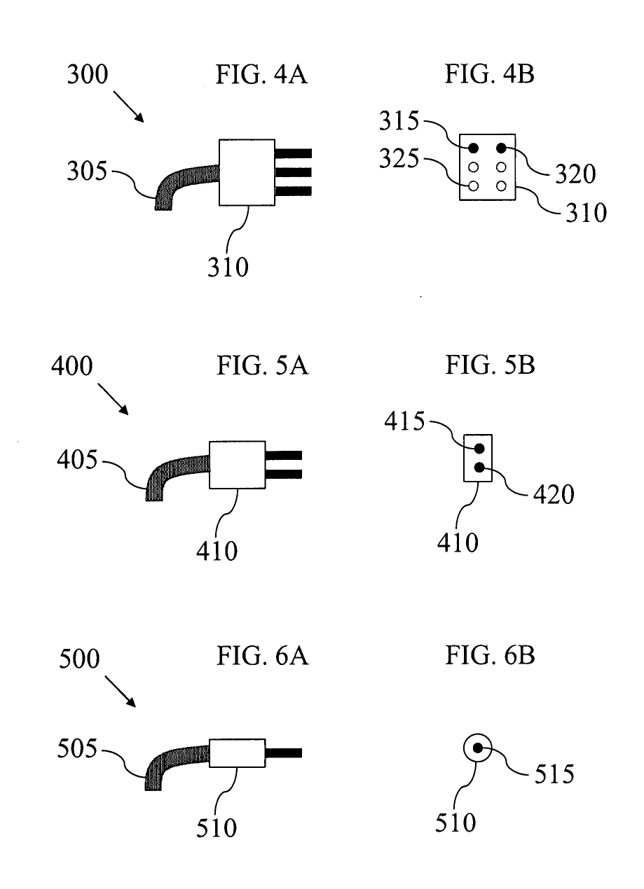

FIG. 4A shows a connector 300 for a detachable handpiece, which connects to a base unit (not shown) by an umbilicus 305. The umbilicus 305 can include conduits to communicate power, signal, and fluid/gas between the base unit and the handpiece. The connector interface 310 attaches to a handpiece. Thus, changing the handpiece requires only removing the handpiece at the end of the umbilicus 305, while the umbilicus 305 remains attached to the base unit.

FIG. 4B shows another view of the connector interface 310, which includes a first pin 315, a second pin 320, and four fluid/gas plugs 325. In various embodiments, the connector interface 310 can also include other plugs. For example, the connector can include a first conduit to deliver power and/or communicate a signal to and/or from the handpiece and a second conduit to communicate a signal from the handpiece to and/or from the base unit. The umbilicus 305 can be permanently attached to the base unit, or can be detachable. The connector 300 does not necessarily have separate power and signal plugs and/or separate power and signal conduits in the umbilicus 305. Rather, the first pin 315 and second pin 320 can be the positive and negative terminals of the power plug. In one embodiment, the pins 315, 320 can function to transport a radio wave signal across the power cords. For example, the signal can be a RF or a X10 signal. However, the invention is not limited to communicating a radio wave signal through two power cords, and can include other means to eliminate the need for a additional signal pins in the connector, e.g. using wireless and/or radio frequency signals.

FIG. 5A shows another connector 400 for a detachable handpiece, which connects to a base unit (not shown) by an umbilicus 405. The umbilicus 405 can include conduits to communicate power and/or signal between the base unit and the handpiece. The connector interface 410 attaches to a handpiece. Thus, changing the handpiece requires only removing the handpiece at the end of the umbilicus 405, while the umbilicus 405 remains attached to the base unit. FIG. 5B shows another view of the connector interface 410, which includes a first pin 415 and a second pin 420. The first pin 415 and a second pin 420, or any two pins of any connector, can be the positive and negative terminals of a connector for transmitting power and/or communicating a signal, or one can transmit power while the second can communicate a signal. One of a pair of pins, or one portion of a multi pin, can be a return for the power and/or the signal.

FIG. 6A shows another connector 500 for a detachable handpiece, which connects to a base unit (not shown) by an umbilicus 505. The umbilicus 505 can include conduits to communicate power and/or signal between the base unit and the handpiece. The connector interface 510 attaches to a handpiece. Thus, changing the handpiece requires only removing the handpiece at the end of the umbilicus 505, while the umbilicus 505 remains attached to the base unit. FIG. 6B shows another view of the connector interface 410, which includes a pin 415. The pin 415, or any pin of any connector, can be a jack plug with two or more contacts such a stereo jack or a coaxial jack. The connector 500 can include one or more plugs for a fluid and/or a gas.

Although FIGS. 4-6 illustrate pin connectors, the invention is not limited to pin connectors and can include virtually any type of connector. For example, the invention includes all ports, plugs, adaptors, fasteners, and the like that establish communication between a conduit in the umbilicus and a conduit in the handpiece, for transmitting signal, energy, fluid, and/or gas between the two conduits.

In various embodiments, the connector includes means for communicating a signal both to and from the handpiece, which can be employed in methods utilizing biofeedback. For example, a user can be prompted for information relating to a biological tissue to be treated. The user can then be provided with one or more treatment parameters for the electromagnetic radiation based on the information. Next, the user can be prompted to trigger a device capable of emitting the electromagnetic radiation to treat the biological tissue. The method can be used iteratively. Furthermore, the parameters for subsequent electromagnetic radiation emissions can be modulated based upon user input, which can be derived from the biological tissue's reaction to the preceding electromagnetic radiation emission.

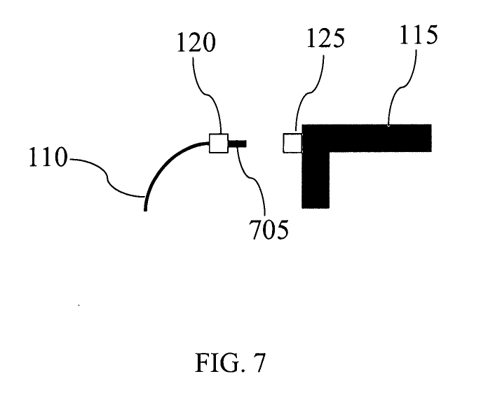

FIG. 7 shows the detachable handpiece 115 disconnected from the umbilicus 110. The detachable handpiece 115 includes a second 125 connector having a first end and a second end. The first end is adapted to detachably connect to an umbilicus 110 having an energy conduit and can receive energy from the energy conduit. The second end is adjacent the handpiece 115 and can communicate energy to the handpiece 115 to drive a source of electromagnetic radiation. The second 125 connector detachably connects the handpiece 115 to an umbilicus 110 through the first 120 connector, and receives energy from a power conduit within the umbilicus 110. The connection between the first 120 connector and the second 125 connector is established through at least one connector pin 705. Although the connector pin 705 shown as associated with the first 120 connector, in various embodiments the first 120 connector can be a male, female, or other type of connector and the second 125 connector can be a mating connector. Once the detachable handpiece 115 is removed, a second detachable handpiece (not shown) can be provided. The second detachable handpiece is suitable for a different treatment or capable of providing different treatment parameters than the handpiece 115.

While the invention has been particularly shown and described with reference to specific embodiments, it should be understood by those skilled in the art that various changes in form and detail may be made therein without departing from the spirit and scope of the invention as defined by the appended claims.

1. A detachable handpiece for treating biological tissue using electromagnetic radiation, the handpiece comprising:
a connector adjacent a first portion of the handpiece, for detachably connecting the handpiece to an umbilicus and for receiving energy from a power conduit in the umbilicus; and
a source of electromagnetic radiation adjacent a second portion of the handpiece and in communication with the connector, for receiving energy from the connector to drive the source of electromagnetic radiation.

2. The detachable handpiece of claim 1 further comprising a controller for at least one of sending and receiving a signal and in communication with the source of electromagnetic radiation, for regulating the source of electromagnetic radiation to deliver electromagnetic radiation to the biological tissue.

3. The detachable handpiece of claim 2 wherein the controller is in communication with the connector, for at least one of sending and receiving a signal transmitted through the connector.

4. The detachable handpiece of claim 2 wherein the signal transmitted through the connector is an RF signal transmitted through the power conduit.

5. The detachable handpiece of claim 2 wherein the controller is adapted for at least one of sending and receiving a wireless signal.

6. The detachable handpiece of claim 2 wherein the controller is adapted for at least one of sending and receiving an electronic signal.

7. A connector for a detachable handpiece for treating biological tissue using electromagnetic radiation, the connector comprising:
a first end adapted to detachably connect to an umbilicus having an energy conduit, the first end for receiving energy from the energy conduit; and
a second end adjacent the handpiece, the second end for communicating energy to the handpiece to drive a source of electromagnetic radiation.

8. The connector of claim 7 wherein the first end is adapted for at least one of sending and receiving a signal from the umbilicus and the second end is adapted for communicating the signal to the handpiece to regulate the source of electromagnetic radiation.

9. The connector of claim 8 wherein the signal is an RF signal transmitted through the power conduit.

10. The connector of claim 8 wherein the signal is an electronic signal transmitted through a second conduit.

11. An apparatus for treating biological tissue using electromagnetic radiation, the apparatus comprising:
an energy source;
an umbilicus connectable to the base unit, the umbilicus having a conduit for transmitting energy from the energy source from a first end to a second end of the umbilicus;
a first connector associated with the second end of the umbilicus;
a second connector detachably connectable to the first connector and for receiving the energy; and
a handpiece associated with the second connector, the handpiece having a source of electromagnetic radiation in communication with the second connector, for receiving the energy to drive the source of electromagnetic radiation.

12. The apparatus of claim 11 further comprising a signal source for providing a signal for regulating the source of electromagnetic radiation.

13. The apparatus of claim 12 further comprising a controller associated with the handpiece and in communication with the signal source and the source of electromagnetic radiation, for regulating the source of electromagnetic radiation.

14. The apparatus of claim 13 wherein the umbilicus is adapted to facilitate communication between the signal source and the source of electromagnetic radiation.

15. The apparatus of claim 14 wherein the signal is an RF signal transmitted through the conduit.

16. The apparatus of claim 13 wherein the signal source is adapted for transmitting a wireless signal and the controller is adapted for at least one of sending and receiving the wireless signal for regulating the source of electromagnetic radiation.

17. The apparatus of claim 14 wherein the signal is an electronic signal transmitted through a second conduit.

18. The apparatus of claim 11 further comprising a controller adapted for regulating at least one property of the energy received by the source of electromagnetic radiation, for regulating the source of electromagnetic radiation.
